(19) United States
(12) Patent Application Publication
(10) Pub. No.: US 2023/0290432 A1
(43) Pub. Date: Sep. 14, 2023

(54) SYSTEM AND METHOD FOR GAINING MECHANISTIC INSIGHTS INTO ACTION OF DRUG USING IN-SILICO TECHNIQUES

(71) Applicant: Innoplexus AG, Eschborn (DE)

(72) Inventors: Om Sharma, Pune (IN); Irfan Yunus Tamboli, Pune (IN); Aryamen Singh, Bareilly (IN)

(73) Assignee: Innoplexus AG, Eschborn (DE)

(21) Appl. No.: 17/688,978

(22) Filed: Mar. 8, 2022

(51) Int. Cl. G16B 5/20; G16B 40/00; G16C 20/60; G16B 35/00

(52) U.S. Cl. CPC G16B 5/20; G16B 40/00; G16C 20/60; G16B 35/00

(57) ABSTRACT

Disclosed is a system for gaining mechanistic insights into action of a drug using in-silico techniques. The system is communicably coupled to a phenotype ontological databank; wherein the system comprises a processor communicably coupled to a memory. The processor is configured to receive a first input of the drug, receive a second input relating to at least one phenotype associated with the drug, identify targets of the drug to obtain a drug target list, determine phenotypic targets of the drug, compare the drug target list with the phenotypic targets of the drug to identify a plurality of overlapping targets therebetween, generate a Drug-Target-Phenotype (DTP) network using the plurality of overlapping targets, compute relevant pathways by performing Signaling Pathway Impact Analysis (SPIA) for the plurality of overlapping targets, generate a Pathway-Target-Phenotype (PTP) network, and compute mechanistic insights into the action of the drug from the analysis of PTP network.

### TECHNICAL FIELD

The present disclosure relates, generally, to gaining mechanistic insights into action of a drug. More specifically, the present disclosure relates to a system and a method for gaining mechanistic insights into action of a drug using in-silico techniques.

### BACKGROUND

Conventionally, process of drug discovery in pharmaceutical industry has always been dependent on a target-based approach for screening of known or unknown drugs. Though the target-based approach has proved to be more successful in discovering follow-up drugs, the discovery of a majority of first-in-class novel drugs requires a phenotypic based approach, thereby enabling to gain mechanistic insights into working mechanisms and action of the drug with respect to multiple phenotypes associated to the drug.

Due to the lack of the mechanistic understanding, gaining mechanistic insights into the drug action in association with the phenotypes of the drug is still a tedious task. Moreover, the already existing phenotypic based approaches are in-vitro techniques, wherein the in-vitro techniques are unable to screen the multiple phenotypes and phenotypic targets at a single, thus both being time consuming, cost and resource extensive. Such approaches might miss out on important phenotypic targets that are significant in deriving certain phenotypes but have not been identified yet.

Therefore, in the light of the foregoing discussion, there still exists a need to overcome the aforementioned drawbacks associated with known techniques for gaining mechanistic insights into action of a drug.

### SUMMARY

The present disclosure seeks to provide a system for gaining mechanistic insights into action of a drug using in-silico techniques. The present disclosure also seeks to provide a method for gaining mechanistic insights into action of a drug using in-silico techniques. An aim of the present disclosure is to provide a solution that overcomes at least partially the problems encountered in the prior art.

In one aspect, an embodiment of the present disclosure provides a system for gaining mechanistic insights into action of a drug using in-silico techniques, the system is communicably coupled to
a phenotype ontological databank comprising information pertaining to a plurality of drugs and the corresponding targets thereof;
   wherein the system comprises a processor communicably coupled to a memory, the processor configured to
receive a name the drug as a first input;
receive a second input relating to at least one phenotype associated with the drug;
fetch targets of at least one existing drug that is similar to the drug to obtain a drug target list;
determine, phenotypes of the drug based on associations between the targets in the drug target list and the phenotypes, said associations being accessed from the phenotype ontological databank;
compare the drug target list with the phenotypic targets of the drug to identify a plurality of overlapping targets therebetween;
generate a network comprising the at least one drug, the targets and the phenotypes;
compute relevant pathways by performing Signaling Pathway Impact Analysis (SPIA) for the plurality of overlapping targets;
generate a Pathway-Target-Phenotype (PTP) network using the most impacted pathways obtained from the results of SPIA;
compute mechanistic insights into the action of the drug from the analysis of PTP network.

In another aspect, an embodiment of the present disclosure provides a method for gaining mechanistic insights into action of a drug using in-silico techniques, wherein the method is implemented using a system communicably coupled to
a phenotype ontological databank comprising information pertaining to a plurality of drugs and the corresponding targets thereof;
   wherein the system comprises a processor communicably coupled to a memory, the method comprising:
receiving a name of the drug as a first input;
receiving a second input relating to at least one phenotype associated with the drug;
fetching targets of at least one existing drug that is similar to the drug to obtain a drug target list;
determining, phenotypes of the drug based on associations between the targets in the drug target list and the phenotypes, said associations being accessed from the phenotype ontological databank;
comparing the drug target list with the phenotypic targets of the drug to identify a plurality of overlapping targets therebetween;
generating a network comprising the at least one drug, the targets and the phenotypes;
computing relevant pathways by performing Signaling Pathway Impact Analysis (SPIA) for the plurality of overlapping targets;
generating a Pathway-Target-Phenotype (PTP) network using the most impacted pathways obtained from the results of SPIA;
computing mechanistic insights into the action of the drug from the analysis of PTP network.

Additional aspects, advantages, features and objects of the present disclosure will be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

### BRIEF DESCRIPTION OF DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those skilled in the art will understand that the drawings are not to scale. Wherever possible, like elements have been indicated by identical numbers.

Embodiments of the present disclosure will now be described, by way of example only, with reference to the following diagrams wherein:

FIG. 1 is a block diagram of a system for gaining mechanistic insights into action of a drug using in-silico techniques, in accordance with an embodiment of the present disclosure;

FIG. 2 is a visual representation of first input of the drug, in accordance with an implementation of the present disclosure;

FIG. 3 is a system for identifying targets of a drug to obtain a drug target list, in accordance with an implementation of the present disclosure;

FIG. 4 is a Pathway-Target-Phenotype (PTP) network, in accordance with an embodiment of the present disclosure; and

FIGS. 5A and 5B collectively illustrate a flowchart depicting steps of a method for gaining mechanistic insights into action of a drug using in-silico techniques, in accordance with an embodiment of the present disclosure.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DETAILED DESCRIPTION OF EMBODIMENTS

The following detailed description illustrates embodiments of the present disclosure and ways in which they can be implemented. Although some modes of carrying out the present disclosure have been disclosed, those skilled in the art would recognize that other embodiments for carrying out or practicing the present disclosure are also possible.

In one aspect, the present disclosure seeks to provide a system for gaining mechanistic insights into action of a drug using in-silico techniques, the system is communicably coupled to
a phenotype ontological databank comprising information pertaining to a plurality of drugs and the corresponding targets thereof;
   wherein the system comprises a processor communicably coupled to a memory, the processor configured to
receive a name the drug as a first input;
receive a second input relating to at least one phenotype associated with the drug;
fetch targets of at least one existing drug that is similar to the drug to obtain a drug target list;
determine, phenotypes of the drug based on associations between the targets in the drug target list and the phenotypes, said associations being accessed from the phenotype ontological databank;
compare the drug target list with the phenotypic targets of the drug to identify a plurality of overlapping targets therebetween;
generate a network comprising the at least one drug, the targets and the phenotypes;
compute relevant pathways by performing Signaling Pathway Impact Analysis (SPIA) for the plurality of overlapping targets;
generate a Pathway-Target-Phenotype (PTP) network using the most impacted pathways obtained from the results of SPIA;
compute mechanistic insights into the action of the drug from the analysis of PTP network.

In another aspect, the present disclosure seeks to provide a method for gaining mechanistic insights into action of a drug using in-silico techniques, wherein the method is implemented using a system communicably coupled to
a phenotype ontological databank comprising information pertaining to a plurality of drugs and the corresponding targets thereof;
   wherein the system comprises a processor communicably coupled to a memory, the method comprising:
receiving a name of the drug as a first input;
receiving a second input relating to at least one phenotype associated with the drug;
fetching targets of at least one existing drug that is similar to the drug to obtain a drug target list;
determining, phenotypes of the drug based on associations between the targets in the drug target list and the phenotypes, said associations being accessed from the phenotype ontological databank;
comparing the drug target list with the phenotypic targets of the drug to identify a plurality of overlapping targets therebetween;
generating a network comprising the at least one drug, the targets and the phenotypes;
computing relevant pathways by performing Signaling Pathway Impact Analysis (SPIA) for the plurality of overlapping targets;
generating a Pathway-Target-Phenotype (PTP) network using the most impacted pathways obtained from the results of SPIA;
computing mechanistic insights into the action of the drug from the analysis of PTP network.

The system and the method of the present disclosure aims to provide a more efficient way to gain mechanistic insights into the working and action of a drug i.e., any substance that is used to prevent, diagnose, treat, or relieve symptoms of a disease or any abnormal condition. Herein, the association of the drug with multiple phenotypes and phenotypic targets is also considered while gaining the mechanistic insights. Furthermore, the system and the method make use of in-silico techniques, that enables the screening of multiple phenotypes and phenotypic targets simultaneously, thereby resulting in precise results while using the system and method for gaining mechanistic insights into the action of a drug. Hence, the system and method in the present disclosure is both time and cost saving.

The system comprises a phenotype ontological databank comprising a plurality of drugs and phenotypic targets corresponding to each of the plurality of drugs thereof. Herein, the phenotype ontological databank uses ontology, which is a data model that represents concepts, attributes, and relationships in the form of a directed acyclic graph. Furthermore, the phenotype ontological databank provides exploratory analysis of microarray and other forms of high-throughput data. Additionally, the phenotype ontological databank is created with the purpose of covering all phenotypic targets related to the plurality of drugs that are top most phenotypic targets for the plurality of drugs. Herein, each individual drug name in the plurality of drugs describes a phenotypic target, such as “kinase activity”. Moreover, the plurality of drugs may have multiple phenotypic targets.

Throughout the present disclosure, the term “processor” refers to a computational element that is operable to respond to and processes instructions that drive the system. Furthermore, the term “processor” may refer to one or more individual processors, processing devices and various elements associated with a processing device that may be shared by other processing devices. Additionally, the one or more individual processors, processing devices and elements are arranged in various architectures for responding to and processing the instructions that drive the system.

Throughout the present disclosure, the term “memory” refers to a volatile or persistent medium, such as an electrical circuit, magnetic disk, virtual memory or optical disk, in which a computer can store data or software for any duration. Optionally, the memory is a non-volatile mass storage such as physical storage media. Furthermore, a single memory may encompass and, in a scenario, wherein the system is distributed, the processing, memory and/or storage capability may be distributed as well.

The system comprises the processor communicably coupled to the memory, wherein the processor is configured to receive a first input of the drug. Herein, the first input corresponds to a form of information associated to the drug which may be provided as the name, two-dimensional (2D) or three-dimensional (3D) structure of the drug, received by the processor to clearly indicate to the system about the specific drug for which the mechanistic insights into the action of the drug is to be gained. In a first example, the first input of the drug received by the processor may be the name of the drug, “Pazopanib”, to indicate the system to gain mechanistic insights into the action of the drug “Pazopanib”. Furthermore, the first input of the drug may be in the form of a Simplified Molecular Input Line Entry System (SMILES), wherein SMILES is a chemical line notation that allows a user to represent a chemical structure in a way that can be used by the processor. Additionally, the first input of the drug may be in the form of a chemical library, wherein a chemical library is a collection of different real stored chemicals and/or virtual chemical compounds containing relevant information, such as for example, but not limited to, chemical structure, purity, quantity and physiochemical characteristics of every compound.

The processor is further configured to receive a second input relating to at least one phenotype associated with the drug. Herein, upon receiving the first input of the disease, the processor receives a second input where the second input comprises of one or more than one phenotype having some association with the drug of interest for which the phenotypic targets are to be screened to further gain mechanistic insights into action of the drug. Herein, the term “phenotype” corresponds to physical form, structure, biological properties, and development processes of an organism. Throughout the present disclosure, the phenotypes are referred to as a set of observable characteristics related to the drug. Continuing from the first example, the second input received by the processor may be “Angiogenesis”, wherein the second input relates to at least one phenotype associated with the first input of the drug. Subsequently, the processor fetches a list of phenotypes which are similar to the at least one phenotype associated with the drug, wherein the similarity of the phenotypes to the at least one phenotype is depicted using a similarity score. Furthermore, the at least one phenotype, such as “Angiogenesis” comprises a phenotype identification (ID), such as “GO:0001525”, wherein the structure is as shown in Table 1

TABLE 1

| Phenotype | | Similarity |
|---|---|---|
| ID | Phenotype name | score |
| GO:0001525 | Angiogenesis | 1 |
| GO:0048514 | Blood vessel morphogenesis | 0.8 |
| GO:0002040 | Sprouting angiogenesis | 0.75 |
| GO:0045765 | Regulation of angiogenesis | 0.75 |
| GO:0016525 | Negative regulation of angiogenesis | 0.75 |
| GO:0001569 | Branching involved in blood vessel | 0.75 |
| | morphogenesis | |
| GO:0045766 | Positive regulation of angiogenesis | 0.75 |
| GO:0048844 | Artery morphogenesis | 0.667 |
| GO:0001570 | Vasculogenesis | 0.667 |
| GO:0061304 | Retinal blood vessel morphogenesis | 0.667 |
| GO:0001568 | Blood vessel development | 0.6 |
| GO:0061042 | Vascular wound healing | 0.6 |
| GO:0002043 | Blood vessel endothelial cell proliferation | 0.6 |
| | involved in sprouting angiogenesis | |
| GO:0002042 | Cell migration involved in sprouting | 0.6 |
| | angiogenesis | |
| GO:1903670 | Regulation of sprouting angiogenesis | 0.6 |
| GO:1903671 | Negative regulation of sprouting angiogenesis | 0.6 |
| GO:1903672 | Positive regulation of sprouting angiogenesis | 0.6 |
| GO:1905555 | Positive regulation of blood vessel branching | 0.6 |
| GO:0035470 | Positive regulation of vascular wound healing | 0.6 |
| GO:0061044 | Negative regulation of vascular wound healing | 0.6 |
| GO:0097070 | Ductus arteriosus closure | 0.571 |
| GO:2001212 | Regulation of vasculogenesis | 0.571 |
| GO:0061156 | Pulmonary artery morphogenesis | 0.571 |

Optionally, the processor is configured to select the second input relating to the at least one phenotype associated with the drug from within a list of phenotypes. Herein, the list of phenotypes is pre-compiled and stored into the system and the processor selects one or more phenotypes from within the pre-compiled list according to the requirements of a user. In case, some particular phenotype according to the requirements of the user is not present in the pre-compiled list, then the processor selects the phenotype which is most similar to the particular phenotype, from within the list of phenotypes.

The processor is configured to fetch targets of at least one existing drug that is similar to the drug to obtain a drug target list. Herein, the target of the drug is usually a protein, which is intrinsically associated with a particular disease process. Furthermore, the target could be addressed by the drug to produce a desired therapeutic effect. Herein, the target is identified and characterized by identifying function of a possible therapeutic agent, wherein the therapeutic agent may be a gene and/or protein and their role in a disease. In this regard, the at least one drug interacts with multiple targets rather than with a single target. Subsequently, the targets that are identified are listed down in a target list, wherein the target list comprises all the targets relevant to the at least one drug given as input to the processor.

Optionally, the processor is configured to use literature mining to fetch drug targets of the known drug. Herein, a majority of new targets are derived from novel biological discoveries first appearing in scientific literature. Herein, sentences are extracted from publications or documents. Furthermore, literature mining uses various keyword mechanisms and countless forms of indexing or document and/or publication classification, as well as straightforward semantic or text search, wherein sets of documents may be retrieved with the help of literature mining, generally with additional refinements such as Boolean combinations of search terms, iterative refinement of searches and so forth, to obtain the majority of new targets. Herein, certain techniques, such as for example, but not limited to, Name Entity Recognition (NER) may be used on scientific literature to identify chemicals, targets, genes, pathways, diseases and utilized with algorithms to procure additional biologically significant words. Thereafter, a plethora of similarity and partitional clustering techniques may be used to group the majority of new targets based on their common terms.

Optionally, the processor is configured to use chemical similarity algorithm to identify the at least one exiting drugs that is similar to the drug and/or unknown drugs. Typically, chemical similarity algorithm is an important methodology used to identify compounds with similar bioactivities based on structural similarity between any two drugs. Herein, the fundamental principle behind the chemical similarity algorithm is chemical similarity principle, which states that if two molecules share similar structures, then they will likely have similar bioactivities. Furthermore, the chemical similarity algorithm most commonly uses approaches that use chemical substructure fingerprints, such as non-hashed structural fingerprints, chemical hashed fingerprints. Typically, in non-hashed structural fingerprints such as Open Babel FP3, each molecule is converted into a binary series of ‘0’ and ‘1’, wherein ‘0’ indicates absence of a particular structure and ‘1’ indicates the presence of the particular substance, so as to compare the chemical similarity between two molecules. Conversely, in chemical hashed fingerprints such as Open Babel FP2, path information is derived from molecular graphs to compare the chemical structures. Thereafter, the chemical similarity is obtained using a distance metric, for instance Tanimoto index and so forth, after procuring chemical fingerprints of the molecules. Moreover, the targets of the drug may be inferred from structured databases with annotated targets sharing highest similarity to the target. Herein, the structured databases may be public bioactivity databases such as for example, but not limited to chemical database maintained by European Bioinformatics Institute of the European Molecular Biology Laboratory (ChEMBL®), PubChem®, DrugBank. If the drug is an unknown drug, then in that case the targets of the drugs which are highly similar to the unknown drug can be considered as the targets of the unknown drug.

Optionally, machine learning algorithm is used to fetch targets of the drug that is unknown. Typically, the machine learning algorithm is a computational approach which can leverage the growing number of large-scale human genomics and proteomics data sets to make in-silico target identification. Herein, machine learning algorithm is used to prioritize the targets according to their similarity to approved drug targets. Notably, the machine learning algorithm predicts the targets of the drug, wherein the drug is an unknown compound. Furthermore, training dataset of the machine learning algorithm may comprise 37,000 compounds and 3000 target information.

Optionally, the processor is configured to use molecular docking method to fetch drug targets of the known drugs and/or unknown drugs. Herein, molecular docking method is bioinformatic modelling that involves interaction of two or more molecules to provide a stable adduct, wherein the term “adduct” refers to a complex that forms when a chemical binds to a biological molecule, such as protein. Subsequently, the molecular docking depends upon binding properties of the targets and ligands of the drug that is unknown and predicts the 3D structure of any complex. Herein, the molecular docking unstructured databases to search for targets, wherein the targets should be in a proper Protein Databank Format (PDB) format. Additionally, the ligand is prepared as a PDB file using software such as Discovery Studio®. Thereby, the ligands are able to organize based upon their ability to interact with given target. Moreover, the molecular docking of small molecules of small molecules to the targets include a pre-defined sampling of possible conformation of the ligand in a particular groove of the targets so to establish an optimized conformation of the complex. Typically, the molecular docking is performed by simulation approach and shape complementarity approach. In particular, high-throughput virtual screening (HTVS) is used for docking many ligands against one or a few receptors, and a combination of pose identification and scoring algorithms constitute foundation of docking engines, including DOCK and AutoDOCK. Furthermore, results of the molecular docking results are evaluated either by visual inspection of the ligand or quantitatively using a scoring algorithm. Herein, HTVS reduces number of intermediate conformations throughout the process of molecular docking, and also reduces thoroughness of final torsional refinement and sampling.

The processor is further configured to determine, phenotypes of the drug based on associations between the targets in the drug target list and the phenotypes, said associations being accessed from the phenotype ontological databank. Herein, phenotypic screening is useful to screen the drug, wherein the drug may be a first-in-class drug, as there is lack of bias while identifying mechanism of action (MOA) of the drug when it is a first-in-class drug. Furthermore, a physiologically relevant biological system or cellular signaling pathway is directly interrogated by chemical matter to identify biologically active compounds. Additionally, phenotypic screening of the drug to procure phenotypic targets aims to modulate production of the proteins with either known human pharmacological activity or a highly validated association with human physiology. Herein, a database such as Innoplexus® Phenotype Ontology Database may be used, wherein the database comprises data from publicly available structured databases such as QuickGo®, Gene Ontology, Human Phenotype Ontology (HPO), Monarch Initiative and so forth. Furthermore, ontologies of phenotypic targets of the drug are taken from datasets of QuickGo®, Gene Ontology, Human Phenotype Ontology (HPO). Additionally, association of diseases to the phenotypic targets are brought in from Monarch Initiative, MalaCards® as well as from unstructured data sources obtained using literature mining. Importantly, the phenotypic ontological databank stores data about ontology of the phenotypes, associated phenotypic targets of the drug and the association of the disease with the phenotype.

The processor is then configured to compare the drug target list with the phenotypic targets of the drug to identify a plurality of overlapping targets therebetween. Herein, correlation of the phenotypic targets of the drug is determined to identify the plurality of overlapping targets, wherein the correlation statistical significance of overlapping is typically determined using hypergeometric distribution. Furthermore, the hypergeometric distribution is used in network-based approaches to identify novel insights for procuring phenotypic targets by identifying overlapping phenotypic targets of the drug.

Continuing from the first example, the at least one phenotype, such as “Angiogenesis” comprises the overlapping target between the at least one phenotype and the drug, that may be for example “[‘VEGFA’, ‘PRKX’, ‘ANG’, ‘EGF’]”. The structure is as shown in Table 2

TABLE 2

| Phenotype | | Overlapping |
|---|---|---|
| ID | Phenotype name | Targets |
| GO:0001525 | Angiogenesis | [‘VEGFA’, |
| | | ‘PRKX’, ‘ANG’, |
| | | ‘EGF’] |
| GO:0001569 | Branching involved in blood vessel | [‘VEGFA’, ‘KDR’] |
| | morphogenesis | |
| GO:0001570 | Vasculogenesis | [“ENG”] |
| GO:0002040 | Sprouting angiogenesis | [“TEK”] |
| GO:0002042 | Cell migration involved in sprouting | [‘AKT1’, |
| | angiogenesis | ‘VEGFA’] |
| GO:0016525 | Negative regulation of angiogenesis | [‘TEK’] |
| GO:0022009 | Central nervous system | [‘ENG’] |
| | vasculogenesis | |
| GO:0035148 | Tube formation | [‘VEGFA’] |
| GO:0045765 | Regulation of angiogenesis | [‘NF1’] |
| GO:0045766 | Positive regulation of angiogenesis | [‘TEK’, ‘KDR’, |
| | | ‘FLT1’, ‘VEGFA’, |
| | | HIF1A’] |
| GO:0060562 | Epithelial tube morphogenesis | [‘PRKX’] |
| GO:0061042 | Vascular wound healing | [‘VEGFA’, ‘KDR¹] |
| GO:0072011 | Glomerular endothelium development | [‘PECAM1’] |
| GO:0090050 | Positive regulation of cell migration | [‘VEGFA’, ‘KDR’] |
| | involved in sprouting angiogenesis | |
| GO:1903589 | Positive regulation of blood vessel | [‘VEGFA’] |
| | endothelial cell proliferation involved | |
| | in sprouting angiogenesis | |
| GO:1903672 | Positive regulation of sprouting | [‘VEGFA’] |
| | angiogenesis | |
| GO:1905065 | Positive regulation of vascular | [‘KIT’] |
| | associated smooth muscle cell | |
| | differentiation | |

The processor is configured to generate a network comprising the at least one drug, the targets and the phenotypes. Herein, the DTP network comprises direct and indirect relation of the drug with the phenotypes and phenotypic targets of the drug. Furthermore, the DTP network are visually represented as simple graphs, with nodes and vertices denoting the drug, the phenotypic targets of the drug and the phenotypes of the drug, and the links or edges denoting the interactions between them. Additionally, the nodes of the DTP network have a number of edges attached to it, wherein the nodes which has maximum number of edges linked to it are important for the integrity of the network. Moreover, the DTP network are modular in nature, wherein a module comprises a set of nodes that are more densely connected with each other than with other nodes in the network.

The processor is configured to compute relevant pathways by performing Signaling Pathway Impact Analysis (SPIA) for the plurality of overlapping targets. Herein, the SPIA takes into account the data about differential expression of genes and furthermore, comprises of the fold change (FC) values, that indicates the magnitude of the upregulation or downregulation change in the gene regulation. Herein, the FC is a measure of the quantity of degree of change between the final relevant pathways of the phenotypic targets and the original relevant pathways of the phenotypic targets. Additionally, FC values are used to perform quantitative analysis of impact on signaling pathways. Herein, the pathways which are most impacted get a highest perturbation (p-pert) score. Moreover, the impact of the pathways is analyzed based on at least two types of data. Herein, firstly, the differentially expressed genes are over-represented in a given pathway as mentioned in the present disclosure. Secondly, abnormal perturbation of the relevant pathway is measured by propagating measured expression changes across pathway topology. Furthermore, the differentially expressed genes which are over-represented in a given pathway is denoted by an independent first probability “P_{NDE}” and the abnormal perturbation of the pathway is denoted by an independent second probability, “P_{PERT}”. Herein, the first probability captures the significance of a given pathway as provided by the over-representation analysis of the number of differentially expressed genes observed on the pathway. Furthermore, value of the “P_{NDE}” represents the probability of obtaining a number of differentially expressed genes on the given pathway at least as large as observed pathway. Herein, the first probability is

P_{NDE}=P(X≥N_{DE}|H_{O})

wherein, H_{O} denotes null hypothesis, wherein the genes that appear as differentially expressed on the given pathway is completely random, N_{DE} denotes number of differentially expressed genes on the pathway analyzed. Notably, the relevant pathways computed for the phenotypic targets using SPIA uses information regarding differentially expressed genes in control with respect to the disease condition only. Moreover, the second probability is calculated based on amount of perturbation measured in each pathway. Thereafter, a global probability value, denoted by “P_{G}” is calculated for the relevant pathways, incorporating parameters, such as the log FC of the differentially expressed genes, statistical significance of set of genes of the pathway and topology of the signaling pathway.

In an embodiment, enriched pathway information of the signaling pathway for the plurality of overlapping targets may comprise a pathway name, such as “MAPK signaling pathway” along with a pathway identification (ID) of the pathway name, that may be for example “hsa04010”, wherein pathway type of the signaling pathway is specified along with gene of the signaling pathway, wherein the pathway type may be for example, “Signal transduction” and the gene of the signaling pathway may be for example, “[‘KIT’]”. Furthermore, output score using SPIA is generated for each of the signaling pathway, wherein the output score comprises the independent first probability “P_{NDE}”, that may be for example, ‘3.70E-35’ for “MAPK signaling pathway”, and the abnormal perturbation of the pathway denoted by the independent second probability, “P_{PERT}”, that may be for example, ‘2’. Thereafter, the global probability value, denoted by “P_{G}” is evaluated for the signaling pathway, that may be for example, ‘5.80E-33’. The structure is as shown in Table 3

TABLE 3

| Enriched pathway information | | | | | | |
|---|---|---|---|---|---|---|
| Pathway | Pathway | Pathway | | SPIA output score | | |
| Name | ID | type | Gene | P_{NDE} | P_{PERT} | P_{G} |
| MAPK | hsa04010 | Signal | [‘KIT’] | 3.70E−35 | 2 | 5.80E−33 |
| signaling | | transduction | | | | |
| pathway | | | | | | |
| HIF-1 | hsa04066 | Signal | [‘EGF’] | 3.50E−25 | 0.48 | 9.80E−24 |
| signaling | | transduction | | | | |
| pathway | | | | | | |
| Phospholipase | hsa04072 | Signal | [‘HIF1A’, | 3.00E−19 | 0:28 | 3.70E−18 |
| D signaling | | transduction | ‘VE GFA’] | | | |
| pathway | | | | | | |
| FoxO | hsa04068 | Signal | [‘EGF’, | 5.60E−13 | 0.28 | 4.80E−12 |
| signaling | | transduction | ‘VEGFA’] | | | |
| pathway | | | | | | |
| Thyroid | hsa04919 | Endocrine | [‘KIT’] | 4.10E−16 | 0.2 | 3.10E−15 |
| hormone | | System | | | | |
| signaling | | | | | | |
| pathway | | | | | | |
| VEGF | hsa04370 | Signal | [‘EGF’] | 9.10E−13 | 0.12 | 3.40E−12 |
| signaling | | transduction | | | | |
| pathway | | | | | | |
| ErbB signaling | hsa04012 | Signal | [‘VEGFA’] | 5.00E−16 | 0.04 | 7.90E−16 |
| pathway | | transduction | | | | |
| Ras signaling | hsa04014 | Signal | [‘HIF1A’] | 6.90E−33 | | |
| pathway | | transduction | | | | |

The processor is then configured to generate a Pathway-Target-Phenotype (PTP) network using the most impacted pathways obtained from the results of SPIA. Herein, interactions of the most impacted pathways based on the perturbation score, with the phenotypic targets is used to generate the network. Furthermore, phenotypes are mapped to the PTP network via association of the phenotype with the phenotypic targets, thus giving rise to the tripartite PTP network. Furthermore, the PTP network are visually represented as simple graphs, with nodes and vertices denoting the pathway, the phenotypic targets of the drug and the phenotypes of the drug, and the links or edges denoting the direction and direction types between them.

The processor is further configured to compute mechanistic insights into the action of the drug from the analysis of PTP network. Herein, the network enables to identify centrality of the targets with respect to the phenotypes or the pathways. Moreover, the PTP network allows to highlight and compare important motifs that involves pathways-targets-phenotypes. Furthermore, the PTP network makes it possible to identify closest path between the first input of the drug and the phenotype, or between the target and the phenotype. Subsequently, such in-depth and precise analysis into the PTP network allows to compute valuable mechanistic insights into the action and working of the drug.

Moreover, the present disclosure also relates to the method as described above. Various embodiments and variants disclosed above apply mutatis mutandis to the method.

Optionally, the method in the present disclosure wherein the method comprises using literature mining to fetch drug targets of known drugs.

Optionally, the method in the present disclosure wherein the method comprises using chemical similarity algorithm to identify the at least one existing drug that is similar to the drug and/or unknown drugs.

Optionally, the method in the present disclosure wherein the method comprises using molecular docking method to predict targets of the at least one drug to obtain the drug target list.

Optionally, the method in the present disclosure wherein the method comprises selecting the second input relating to at least one phenotype associated with the drug from within a list of phenotypes.

Optionally, the method in the present disclosure wherein the method comprises performing Signaling Pathway Impact Analysis (SPIA) using differential expression analysis of the plurality of overlapping targets.

### DETAILED DESCRIPTION OF DRAWINGS

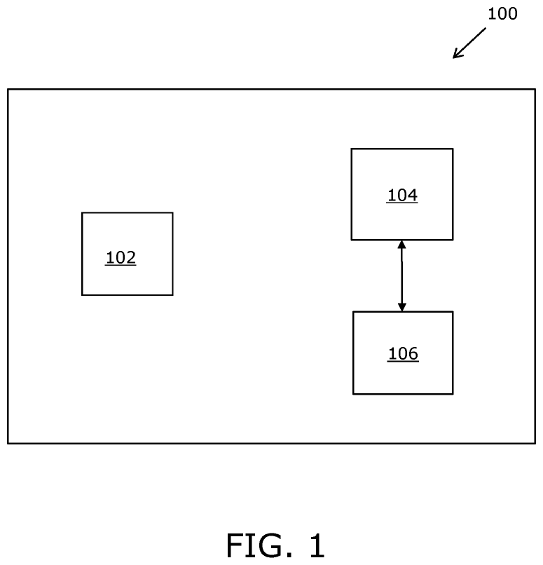

Referring to FIG. 1, there is shown a block diagram of a system 100 for gaining mechanistic insights into action of a drug using in-silico techniques, in accordance with an embodiment of the present disclosure. Herein, the system 100 comprises a phenotype ontological databank 102, wherein the phenotype ontological databank 102 comprises a plurality of drugs and the corresponding targets thereof. Furthermore, the system 100 comprises a processor 104 communicably coupled to a memory 106.

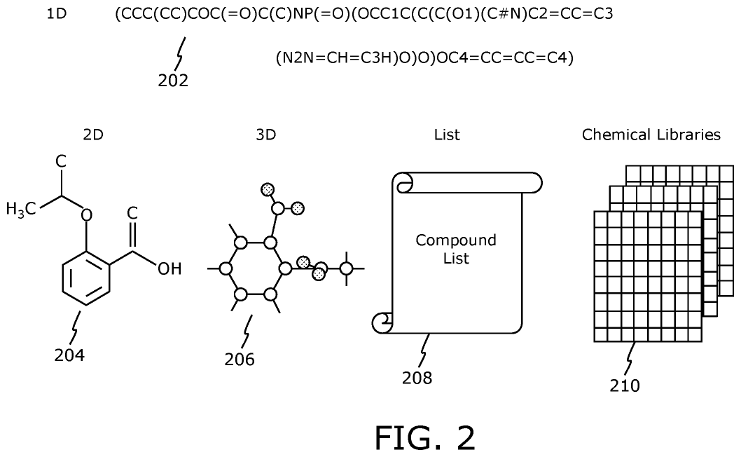

Referring to FIG. 2, there is shown a visual representation of a first input of a drug, in accordance with an implementation of the present disclosure. Herein, the first input of the drug may be in the form of a Simplified Molecular Input Line Entry System (SMILE) 202, wherein SMILES is a chemical line notation that allows a user to represent a chemical structure. Furthermore, the first input of the drug may be in the form of a two-dimensional (2D) chemical structure 204 or a three-dimensional (3D) chemical structure 206, a compound list 208 or as a chemical library 210.

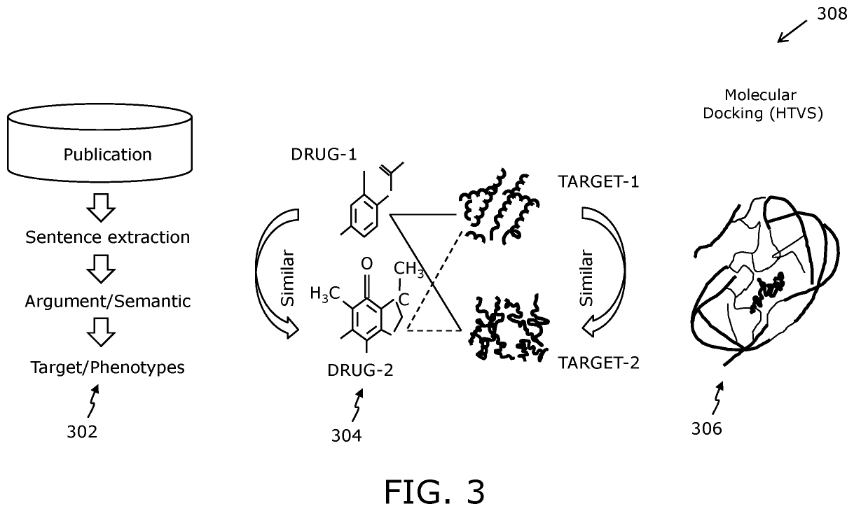

Referring to FIG. 3, there is shown a system 300 for identifying targets of at least one drug to obtain a drug target list, in accordance with an implementation of the present disclosure. Herein, literature mining 302 is used to fetch targets of the at least one drug from publications. Subsequently, sentence extraction is executed using semantic search and consequently the targets of the at least one drug is identified. Optionally, chemical similarity algorithm 304 is used to fetch targets of the at least one drug that is known. Herein, the chemical similarity algorithm 304 identifies compounds with similar bioactivities based on structural similarity between a first drug “Drug 1” and a second drug “Drug 2”. Additionally, targets such as “Target 1”; and “Target 2”, may be inferred with annotated targets sharing highest similarity. Optionally, molecular docking method 306 is used to fetch targets of the at least one drug that is unknown. Herein, molecular docking method 306 is a bioinformatic modelling that involves interaction of two or more molecules to provide a stable adduct, wherein the term “adduct”; refers to a complex that forms when a chemical binds to a biological molecule, such as protein.

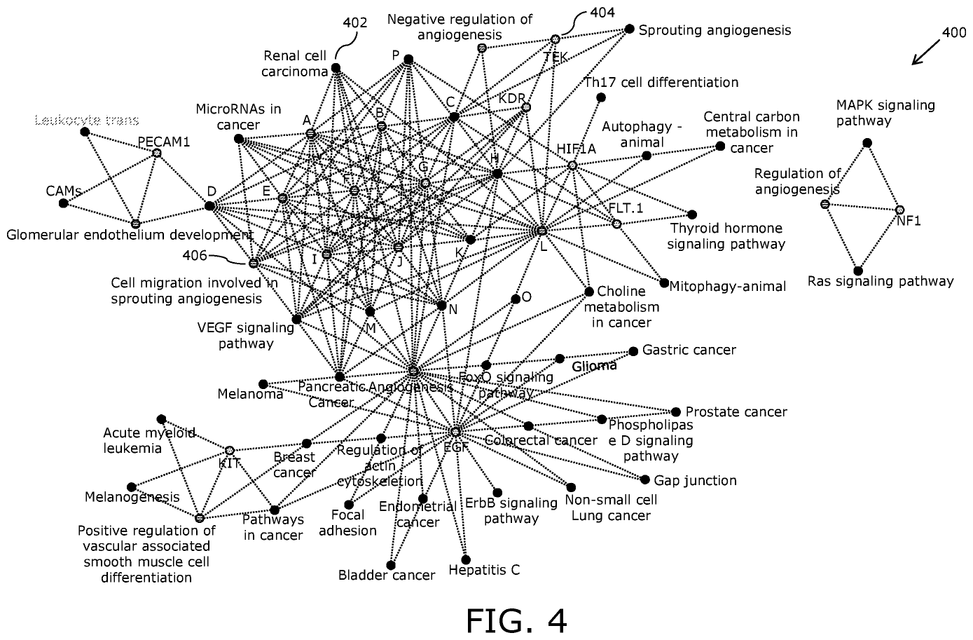

Referring to FIG. 4, there is shown a Pathway-Target-Phenotype network (PTP) 400, in accordance with an embodiment of the present disclosure. Herein, the PTP network 400 comprises direct and indirect relation to at least one pathway 402 with phenotypic targets 404 and phenotypes 406 in association with the disease. Furthermore, the PTP network 400 are visually represented as simple graphs, with nodes and vertices, wherein the nodes have a number of edges attached to it. The nodes of the PTP network are labelled as shown in Table 4

TABLE 4

| LABEL | NODE |
|---|---|
| A | “positive regulation of sprouting angiogenesis” |
| B | “vascular wound healing” |
| C | “Rheumatoid arthritis” |
| D | “Fluid shear stress and atherosclerosis” |
| E | “Tube formation” |
| F | “Branching involved in blood vessel morphogenesis” |
| G | “VEGFA” |
| H | “HIF-1 signaling pathway” |
| I | “Positive regulation of cell migration involved in sprouting |
| | angiogenesis” |
| J | “Branching involved in blood vessel morphogenesis” |
| K | “Proteoglycans in cancer” |
| L | “Positive regulation of angiogenesis” |
| M | “Human cytomegalovirus infection” |
| N | “Human Papillomavirus infection” |
| O | “PD-L1 expression and PD-1 checkpoint pathway in cancer” |
| P | “Kaposi-sarcoma associated herpesvirus infection” |

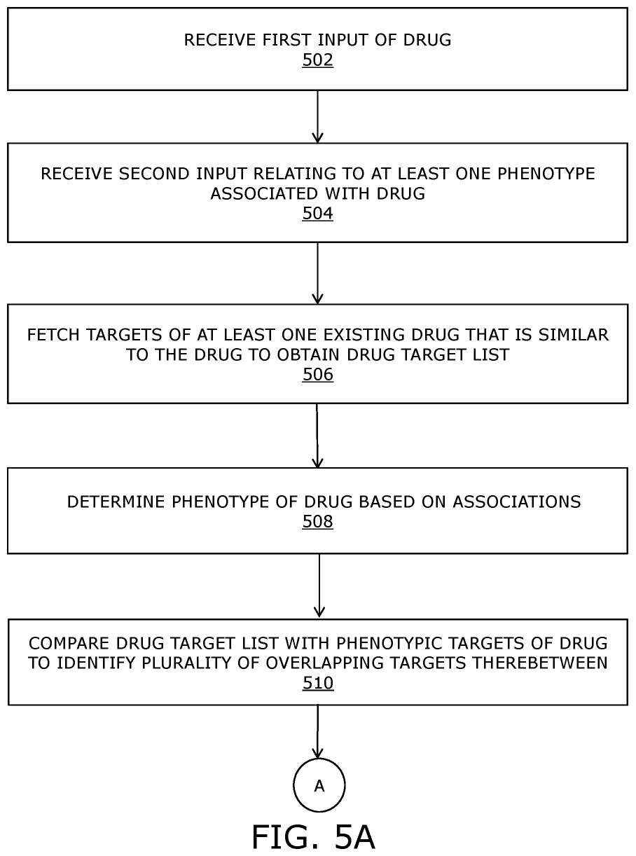

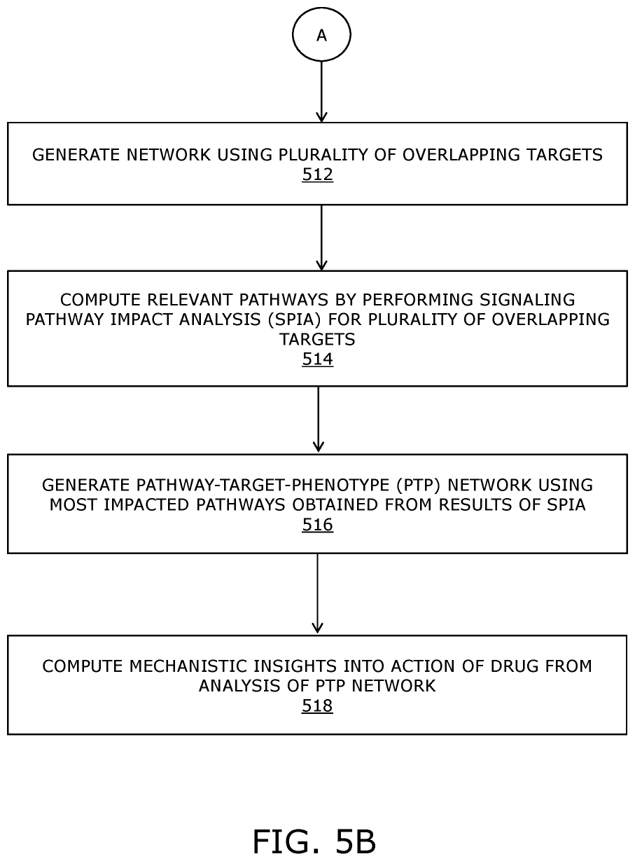

Referring to FIGS. 5A and 5B collectively, there is shown a flowchart depicting steps of a method for gaining mechanistic insights into action of a drug using in-silico techniques, in accordance with the embodiments of the disclosure. At step 502, a first input of the drug is received. At step 504, a second input relating to at least one phenotype associated with the drug is received. At step 506, targets of the drug are fetched to obtain a drug target list. At step 508, phenotype targets of the drug based on associations between the targets in the drug target list and the phenotypes, said associations being accessed from the phenotype ontological databank. At step 510, the drug target list is compared with the phenotypic targets of the drug to identify a plurality of overlapping targets therebetween. At step 512, a network using the plurality of overlapping targets is generated. At step 514, relevant pathways are computed by performing Signaling Pathway Impact Analysis (SPIA) for the plurality of overlapping targets. At step 516, a Pathway-Network-Phenotype (PTP) network using the most impacted pathways obtained from the results of SPIA is generated. At step 518, mechanistic insights into action of the drug are computed from analysis of the PTP network.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural.

1. A system for gaining mechanistic insights into action of a drug using in-silico techniques, the system is communicably coupled to
a phenotype ontological databank comprising information pertaining to a plurality of drugs and the corresponding targets thereof;
wherein the system comprises a processor communicably coupled to a memory, the processor configured to
receive a name the drug as a first input;
receive a second input relating to at least one phenotype associated with the drug;
fetch targets of at least one existing drug that is similar to the drug to obtain a drug target list;
determine, phenotypes of the drug based on associations between the targets in the drug target list and the phenotypes, said associations being accessed from the phenotype ontological databank;
compare the drug target list with the phenotypic targets of the drug to identify a plurality of overlapping targets therebetween;
generate a network comprising the drug, the targets and the phenotypes;
compute relevant pathways by performing Signaling Pathway Impact Analysis (SPIA) for the plurality of overlapping targets;
generate a Pathway-Target-Phenotype (PTP) network using the most impacted pathways obtained from the results of SPIA;
compute mechanistic insights into the action of the drug from the analysis of PTP network.

2. A system of claim 1, wherein the processor is configured to use literature mining to fetch drug targets of known drugs.

3. A system of claim 1, wherein the processor is configured to use chemical similarity algorithm to identify the at least one existing drug that is similar to the drug and/or unknown drugs.

4. A system of claim 1, wherein the processor is configured to use molecular docking method to predict targets of the at least one drug to obtain the drug target list.

5. A system of claim 1, wherein the processor is configured to select the second input relating to at least one phenotype associated with the drug from within a list of phenotypes.

6. A system of claim 1, wherein the processor is configured to perform Signaling Pathway Impact Analysis (SPIA) using differential expression analysis of the plurality of overlapping targets.

7. A computer-implemented method for gaining mechanistic insights into action of a drug using in-silico techniques, wherein the method is implemented using a system communicably coupled to
a phenotype ontological databank comprising information pertaining to a plurality of drugs and the corresponding targets thereof;
wherein the system comprises a processor communicably coupled to a memory, the method comprising:
receiving a name of the drug as a first input;
receiving a second input relating to at least one phenotype associated with the drug;
fetching targets of at least one existing drug that is similar to the drug to obtain a drug target list;
determining, phenotypes of the drug based on associations between the targets in the drug target list and the phenotypes, said associations being accessed from the phenotype ontological databank;
comparing the drug target list with the phenotypic targets of the drug to identify a plurality of overlapping targets therebetween;
generating a network comprising the drug, the targets and the phenotypes;
computing relevant pathways by performing Signaling Pathway Impact Analysis (SPIA) for the plurality of overlapping targets;
generating a Pathway-Target-Phenotype (PTP) network using the most impacted pathways obtained from the results of SPIA;
computing mechanistic insights into the action of the drug from the analysis of PTP network.

8. A method of claim 7, wherein the method comprises using literature mining to fetch drug targets of known drugs.

9. A method of claim 7, wherein the method comprises using chemical similarity algorithm to identify the at least one existing drug that is similar to the drug and/or unknown drugs.

10. A method of claim 7, wherein the method comprises using molecular docking method to predict targets of the at least one drug to obtain the drug target list.

11. A method of claim 7, wherein the method comprises selecting the second input relating to at least one phenotype associated with the drug from within a list of phenotypes.

12. A method of claim 7, wherein the method comprises performing Signaling Pathway Impact Analysis (SPIA) using differential expression analysis of the plurality of overlapping targets.
